(19) United States
(12) Patent Application Publication
(10) Pub. No.: US 2022/0300908 A1
(43) Pub. Date: Sep. 22, 2022

(54) SYSTEM AND METHOD FOR CLAIM REIMBURSEMENT

(71) Applicant: Claimwell Technologies Inc., Vancouver (CA)

(72) Inventor: Ricci Plotkin, Vancouver, CA (US)

(21) Appl. No.: 17/696,536

(22) Filed: Mar. 16, 2022

Related U.S. Application Data
(60) Provisional application No. 63/161,683, filed on Mar. 16, 2021.

(51) Int. Cl. G06Q 10/10; G16H 10/60

(52) U.S. Cl. CPC G06Q 10/1057; G16H 10/60

(57) ABSTRACT

Systems and methods are described for managing a claim on behalf of a provider. Based on the claim and a payment setting of the payer, a reimbursement matrix is generated, and a request for payment is created with the reimbursement matrix and transmitted to the payor. In particular embodiments, the reimbursement matrix is a pre-coded reduction schedule wherein for each one of a plurality of predetermined dates over a payment period, the outstanding payment amount is discounted from the full claim amount by an amount that decreases over time. If the claim has not yet been settled, a notification of the outstanding payment amount is sent to the payer on each one of the plurality of predetermined dates. Particular embodiments also streamline and automate the process of collecting explanation/verification of benefits, medical records/reports and primary care physician reports to allow the payor to adjudicate the claim.

### REFERENCE TO RELATED APPLICATIONS

This application claims priority from U.S. Patent Application No. 63/161,683 filed on Mar. 16, 2021 entitled “SYSTEM AND METHOD FOR CLAIM REIMBURSEMENT”. This application claims the benefit under 35 U.S.C. § 119 of U.S. Patent Application No. 63/161,683 filed on Mar. 16, 2021 entitled “SYSTEM AND METHOD FOR CLAIM REIMBURSEMENT” which is incorporated herein by reference in its entirety for all purposes.

### TECHNICAL FIELD

The present invention relates to claims management systems. Particular embodiments relate to claim reimbursement for health care-related claims.

### BACKGROUND

Medical billing systems suffer from problems such as cost inefficiencies, delays, non-user friendly experiences and siloed information. In general, most software solutions for medical billing systems are designed for payers (e.g. insurance providers) rather than for the service providers (e.g. hospitals or medical centers). The software solutions that are designed for providers, such as in the RCM (Revenue Cycle Management) or EHR/EMR (Electronic Health Record or Electronic Medical Record) industries, are focused on document exchange (e.g. sending/receiving documents such as medical records) or on clinical, scheduling, or billing components of the value chain.

In many countries, including in particular the United States of America, healthcare payment systems are complicated and fragmented. Further complications can arise when out-of-network patients, such as travelers, receive medical treatment, since they are generally billed much higher fees than in-network patients who receive the same type of services. Healthcare payment systems often involve layers of middle actors with diametrically opposed interests. For example, a provider may engage a third party agent to collect payment on a claim. This agent may receive a commission based on how much they collect for the provider. Conversely, payers may engage agents who receive a commission based on how much they save (i.e. the discount). Payers and their agents are continuously finding ways to minimize their payments (e.g. using reference-based pricing tactics).

As such, there is a need for improved systems and methods to manage claims in a more efficient manner, to ensure fair and timely payment of claims. There is a need for systems and methods for claims management that benefit both the provider and the payer.

### SUMMARY OF THE DISCLOSURE

Aspects described herein are directed to systems and methods for medical providers to send healthcare claims to payers, produce defensible reimbursement options for non-contracted emergency services, facilitate accurate document, communication and payment exchange, in a secure cloud environment with tracking and audit functionality.

One aspect relates to a method for managing a claim on behalf of a provider. The method includes receiving the claim via a claim input form provided through a device operated by the provider. The claim includes patient-identifying information for a patient and claim information. The method includes identifying from the claim a payer associated with the patient; based on the claim and a payment setting of the payer, generating a reimbursement matrix, and creating a request for payment with the reimbursement matrix; and transmitting the request for payment to the provider. In particular embodiments, the reimbursement matrix is generated by calculating a full claim amount from the claim information, and determining, for each one of a plurality of predetermined dates over a payment period, an outstanding payment amount, wherein the outstanding payment amount is discounted from the full claim amount by an amount that decreases over time. The payment setting determines, for each of the predetermined dates over the payment period, an extent to which the claim amount is discounted.

If the claim has not yet been settled, a notification of the outstanding payment amount is sent to the payer on each one of the plurality of predetermined dates. When the payer elects to settle the claim, payment information is collected from the payer.

Particular embodiments streamline and automate the process of collecting information and documents by the payer to adjudicate the claim. These documents include: explanation of benefits or verification of benefits information, medical records and/or a medical report, and primary care physician records.

Particular embodiments include an analytics service to track the payment performance and payment patterns of payers. This information can be used to present recommendations to the provider for claims management.

Additional aspects of the present invention will be apparent in view of the description which follows.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 is a system architecture diagram for a claims management platform according to one embodiment;

FIG. 2A is a screenshot of a reimbursement matrix that may be used in the claims management platform of FIG. 1;

FIG. 2B is a flowchart of a process for account set-up by a provider;

FIG. 3A is a flowchart of a process for submitting a pre-claim by a provider, according to one embodiment;

FIG. 3B is a flowchart of a process for submitting a claim by a provider;

FIG. 3C is a screenshot of a sample claim page displayed to the payer;

FIG. 4 is a flowchart of a process for viewing a claim, requesting information and paying a claim by a payer;

FIG. 5 is a flowchart of a process for tracking a claim by a provider; and

FIG. 6 is a screenshot of a sample claim page displayed to the provider.

### DETAILED DESCRIPTION

The description, which follows, and the embodiments described therein, are provided by way of illustration of examples of particular embodiments of the principles of the present invention. These examples are provided for the purposes of explanation, and not limitation, of those principles and of the invention.

The present disclosure relates to a claims management system. Particular embodiments are directed to a claim reimbursement platform for health care-related claims. Providers of health care services, such as hospitals, medical centers, ambulance services, and medical practitioners, and other health care providers, can use the system to generate, manage, and obtain payment on a claim. Payers receive a bill from the provider through the claims management system. Payers may include commercial payers such as private health insurers or employers, private organizations or individuals, or government (e.g. for a publicly-funded health care plan). To incentivize more timely payment the system establishes a reimbursement matrix. The matrix may be in the form of a pre-coded reduction schedule wherein a maximum discount is applied to the claim amount if the payer pays by a predetermined date, such as within 5 days, or within 15 days. As time passes, the discount applied to the claim amount decreases. The discount decreases until it reaches zero or a minimal discount by the payment deadline. Beyond that date, interest charges may start accruing. The system sends automated notifications and payment reminders to the payer. The system facilitates collection of documents by the payer, such as medical records and primary care physician (PCP) records, which can be used by the payer to adjudicate the claim. The system also facilitates electronic payment by the payer.

After a predetermined end date (such as 150 days from the initial request for claim payment, or 200 days from the initial request for claim payment), the claims may be transmitted to a third party program for collection through other means (e.g. via the court system).

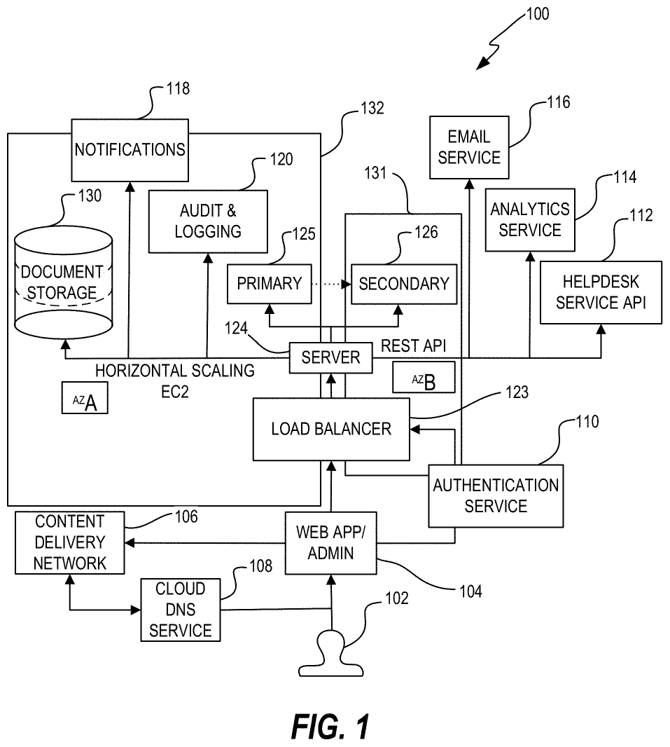

FIG. 1 is a system architecture diagram for a claims management platform according to one embodiment. FIG. 1 shows the overall system 100, which is accessed by a user 102 via a portal provided through a user interface 104 on a client device. The platform is accessed by both providers and payers. As such, user 102 may be an administrator or other user of a provider account, or an administrator or other user of a payer account.

In the illustrated embodiment, the claims management platform includes a plurality of web services, such as an authentication service 110, a helpdesk service API 112, analytics service 114, email service 116, notifications service 118, and audit and logging service 120. Each service provides a function on the platform, as described in further detail below. Audit and logging service 120 and analytics service 114 may be used to ensure compliance with health information privacy laws and other regulatory requirements. It may also be used to confirm payer, provider and system activities so that potential legal remediation for unpaid or underpaid claims can be accomplished with a full picture of what has taken place.

In the illustrated embodiment of FIG. 1, system 100 makes use of a content delivery network (CDN) 106 and cloud Domain Name Server (DNS) service 108. CDN 106 reduces latency in loading content to a web page that is viewed by user 102 to access various functions of the system. DNS service 108 routes the end user requests to the web applications.

System 100 includes a group of backend servers 124, and a load balancer 123 for efficiently distributing user requests among the servers 124. In addition, system 100 includes a primary database server 125 and a secondary database server 126. Data is replicated between the servers 125, 126, such that user 102 can be redirected to the secondary database server 126 if the primary database server 125 becomes unavailable. Database servers 125, 126 store and manage data that is used by system 100 to generate, track, manage, and collect payment on claims. In addition, system 100 may include a database for document storage 130. Document storage 130 may be used for storing provider billing documents, medical records, itemized statements, and insurance documents such as policy benefit letters (explanation of benefits), and denial documents.

In some embodiments the infrastructure of the platform is arranged as a plurality of availability zones, or logical data centers with their own power and networking. The illustrated embodiment of FIG. 1 has availability zones 131, 132. The platform may be structured so that if one of these availability zones 131, 132 were to go down, this would not impact the operation of the other availability zone(s).

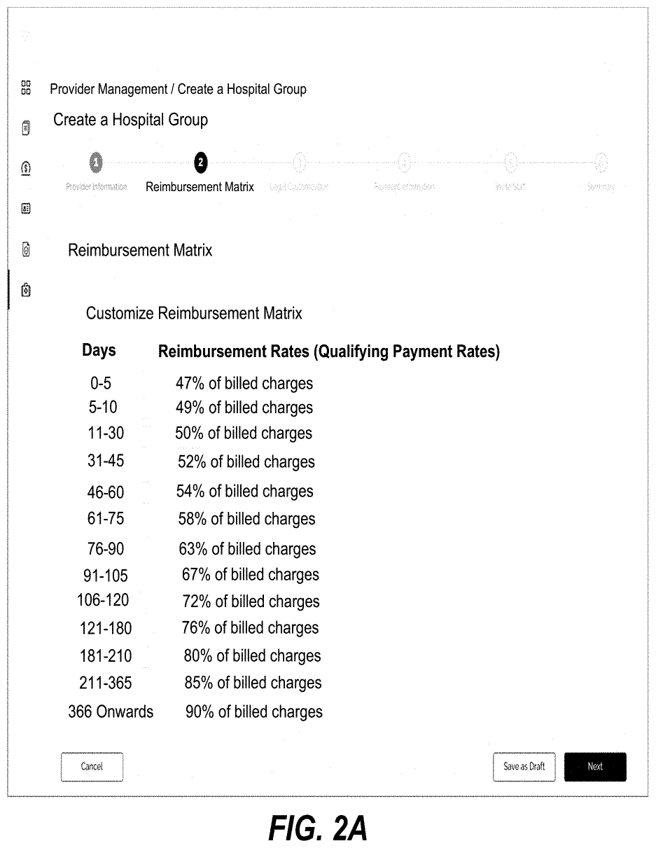

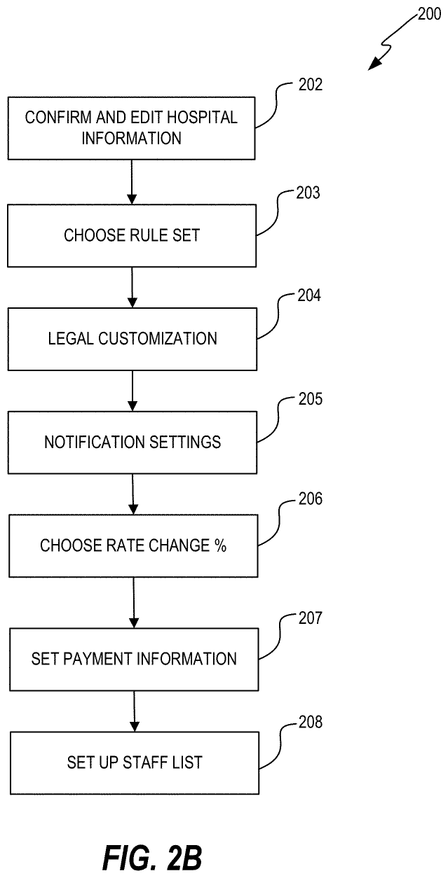

In the system described herein, a reimbursement matrix is generated to incentivize early settlement of a claim by setting a reimbursement schedule with decreasing discounts over time. Thus, the earlier the payer settles the claim, the greater the discount on the claim, and the greater the amount saved by the payer. The reimbursement matrix is customizable by the provider in some embodiments. FIG. 2A is a screenshot of an exemplary reimbursement matrix which is customizable by the provider. In the illustrated example, if payment is made within 0-5 days of invoicing, the payment required is 47% of the billed charges (i.e. 53% discount on the billed charges). If payment is made within 5-10 days, the payment increases to 49% of the billed charges (i.e. 51% discount on the billed charge). This payment increases (discount decreases) until 366 days and onward, at which point the payment required is 90% of the billed charges (i.e. 10% discount on the billed charges), and interest charges start accruing. The provider may customize the schedule and/or the discount by entering different values in each row of the “Days” and “Reimbursement Rates (Qualifying Payment Rates)” columns. In particular embodiments, the reimbursement matrix is further customizable for different classes of payers (e.g. based on payment performance) and different classes of patients (e.g. based on medical records and/or how often the patient requires medical care), and/or other factors.

FIG. 2B shows a process 200 for account set-up by a provider according to one embodiment. In order to set up an account to use the platform of FIG. 1, the user 102 (which is the provider in this case) must have the necessary credentials to access the platform. Setting up user access and verifying user credentials may be performed through authentication service 110 of FIG. 1. For example, to set up user access, authentication service 110 may send an email to the user containing a link to create their account. After clicking on the link the user is taken to a web application 104 which prompts the user to confirm their email address and set a password. The user uses their email address and password to log on to the platform. Once the user has been authenticated and is logged in, process 200 begins at block 202 by the user confirming and editing the provider's information (e.g. hospital, health center or physician's office information).

Process 200 then proceeds to block 203 by prompting the user to choose a rule set which determines the initial discount rate for the reimbursement matrix. The rule sets may be aggressive (e.g. start with 30% discount for payment on day 1), middle (e.g. start with 40% discount for payment on day 1), and fair (e.g. start with 50% discount for payment on day 1). In general, the billed charges may be based on a fixed charge master, which varies between different health care providers. Certain hospitals or other health care providers have lower prices on their charge master, so for those providers it may be desirable to start with a lower discount rate (e.g. 30%) and therefore be more aggressive with respect to payment collection.

After choosing a rule set, process 200 proceeds to prompt the user to customize legal settings at block 204 (e.g. choose whether the provider or the operator of the platform should handle the payment collection after the payment deadline has passed), and notification settings at block 205 (e.g. send notification to provider every X days, and send notification to patient after Y days). In addition, process 200 invites the user to choose the rate percentage at block 206 (e.g. setting a 3% rate change means that the discount drops by 3% for each predetermined period; such predetermined period may be set to a default 15 days), set its payment information at block 207 (e.g. banking, wire transfer details, and finance contact information), and set up the provider's staff list at block 208. Each staff member may be assigned a role and may be invited to set up their own user account under the provider so as to be able to access the platform to perform various functions. Functions may vary in accordance with the user's role and may include generating claims, tracking or viewing claims, etc.

In a particular embodiment, two types of claims may be generated by the provider: (a) a pre-claim, generated before the patient has completed their treatment), and (b) a regular claim, generated once the patient has completed their treatment and is discharged. Pre-claims may be useful for larger or more contentious claims, as they permit the insurer (the payer) to assess, in advance of the completion of the treatment, the payment amount they need to reserve.

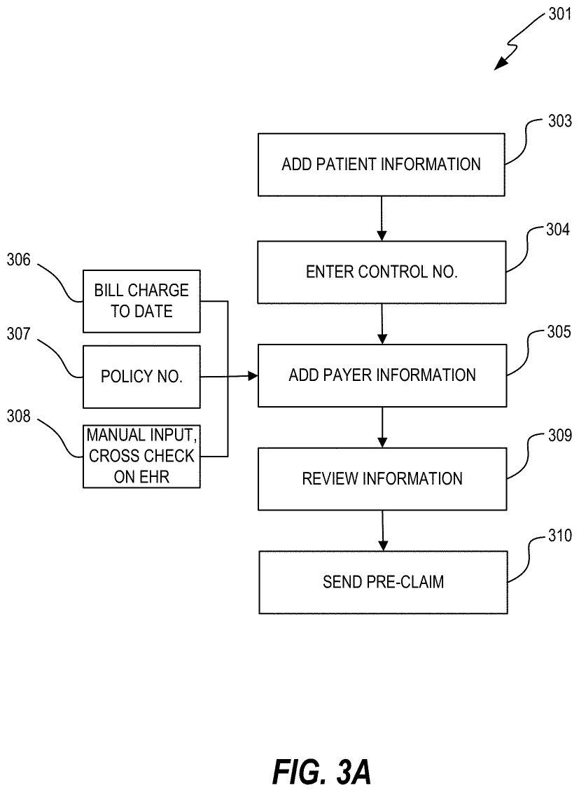

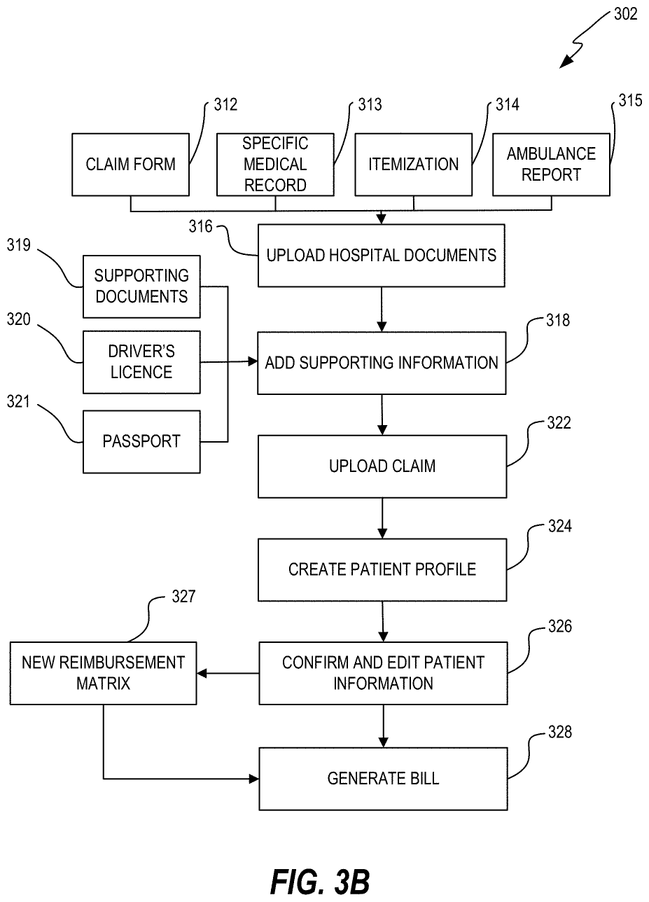

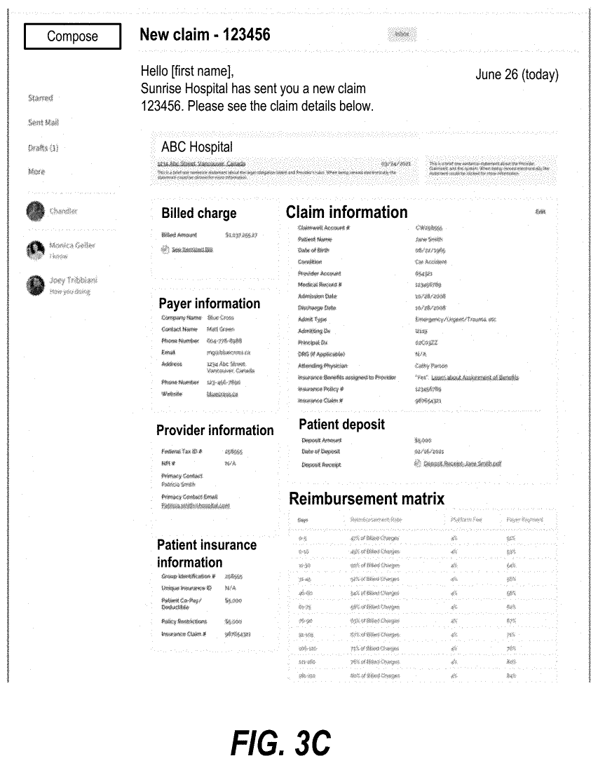

FIG. 3A is a flowchart of a process 301 for a provider to submit a pre-claim, according to one embodiment. Process 301 is started once the patient has been admitted to into the hospital (i.e. the provider). The user logs into the system, and begins at block 303 by entering the patient information and uploading available documents, such as medical records and medical reports. At block 304 the user enters the control number (a number which uniquely identifies the claim). At block 305 the user enters payer (insurer) information such as a bill charge to date 306 (for expenses incurred already by the client), insurance policy number 307, and manual claim input 308 and results of a cross check on electronic health records. The user reviews the pre-claim information at block 309, and sends the pre-claim at block 310 to the payer. Manual claim input 308 enables the provider to manually input an offer to the payer based on the interim billed charge. If the provider believes there is a risk that the provider will deny payment, for example, due to pre-existing conditions, then the provider may choose to offer the payer an incentive to pay the claim, by way of a greater than usual financial reduction for the claim. This is a form of claim denial management.

Pre-claims are eventually converted to regular claims once the patient is discharged. In most cases, pre-claims are not created, and only a regular claim is submitted. FIG. 3B is a flowchart of a process 302 for submitting a claim by a provider according to one embodiment. By the time process 302 is started, the patient is generally already discharged from the hospital (i.e. the provider). The user logs into the system, and begins at block 316 by uploading available documents, such as a standardized claim form 312 (e.g. UB-04), specific medical records 313, itemized bill 314, and ambulance report 315. At block 318 the user inputs supporting information such as supporting documents 319 (e.g. health insurance documentation), driver's license 320, and passport 321. The user uploads the claim at block 322, and creates a patient profile at block 324. The patient profile may include patient information and a reason for attendance. The reason for attendance may be obtained through an Application Programming Interface (API) which links to a particular website to look up a verbal description for a condition using the alphanumeric code indicated on the claim form 312. Patient information is then confirmed, and edited if necessary, at block 326.

In the illustrated embodiment, a reimbursement matrix is retrieved at block 327 and used to generate a bill at block 328 with a reimbursement payment schedule. As previously discussed, in accordance with the reimbursement matrix an initial discount is applied to the billed charges, which discount decreases over time. In other embodiments, rather than a reimbursement matrix, the actual billed charges, or a government-determined benchmark charge, may be displayed in the bill.

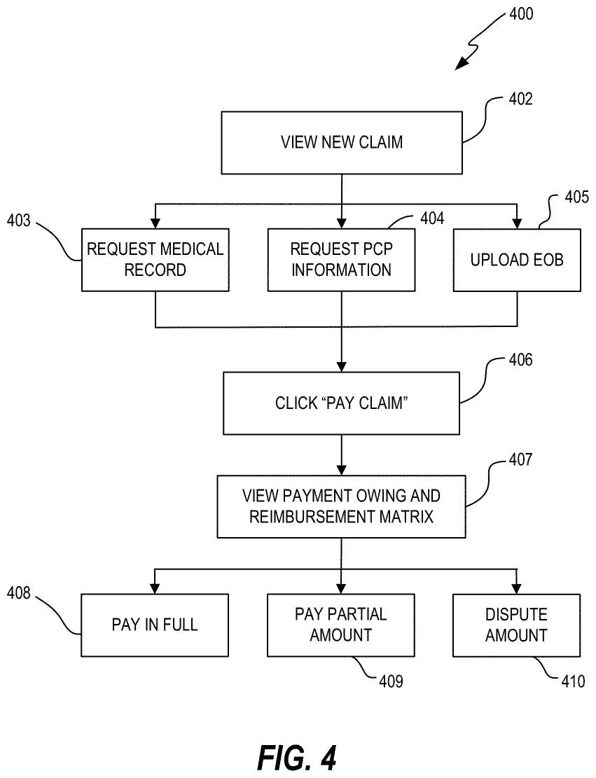

This bill is transmitted to the appropriate payer, which may be the patient's health care insurer where the patient is covered under a health plan. Email service 116 may be used to send the payer an email with a link to view and pay the claim. A screenshot of an exemplary claim information sheet that is displayed to the payer is shown in FIG. 3C. This information sheet includes the patient information and reason for attendance. The payer may also be able to view any medical record and medical report of the patient that is attached to the claim. In particular embodiments, where no medical record or medical report is attached with the claim, the payer may have the option to click a button to request these documents from the provider, as described below with reference to FIG. 4.

The information sheet of FIG. 3C displays the payment schedule which has payment amounts calculated in accordance with the reimbursement matrix. The information sheet also displays a co-pay amount where it is determined that the patient's insurance plan with the payer does not cover all of the billed charges. Where available, information concerning the co-pay amount is provided by the patient or the payer (insurer) during the time of service; otherwise, it can be added later. This co-payer may be the patient himself/herself or another private insurer where the patient has coverage under an additional insurance plan.

The provider may choose to send the regular claim to the payer (insurer) as is, or they can include an additional “make an offer” function. For example, certain types of claims (such as for those that may be deemed to be caused by underlying conditions) are more likely to be denied reimbursement by the insurer, resulting in the patient having the pay the bill. In such cases the provider may want to present a deal to the payer given that collecting payment from a patient can present challenges, particularly if the patient is out-of-network. Even though the claim may be considered to be outside the scope of the insurance coverage, the payer may be motivated to reimburse the claim at a discounted rate to avoid negative publicity for denying a claim.

FIG. 4 is a flowchart of a process 400 according to one embodiment for a payer to view a claim, request information, and pay the claim. Once a claim has been created by a provider, email service 116 sends an email to the payer with a link to access the claim. In addition, the payer is required to login to the system using their email address and password to view the claim at block 402. The claim view may be in the form of an information sheet such as that shown in FIG. 3C. Where no medical record or medical report is attached with the claim, the payer may have the option at block 403 to click a button to request these documents from the provider. In addition, the payer will have the option to request Primary Care Physician (PCP) records at block 404 from the patient's primary care physician. These requests are auto-generated in electronic communication form (e.g. email, facsimile) which are transmitted to the provider and PCP. In response to receiving these requests, the requested documents are transmitted by the provider and the PCP to the payer. In addition, at block 405 the payer uploads the plan's Explanation of Benefits (EOB) which lists policy exclusions and inclusions. The patient's PCP records may be reviewed by the payer to asses whether the patient has any underlying conditions and whether there is other information which would affect claim adjudication. Conventionally, there are often delays associated with gathering medical reports and PCP records, since telephone communications and letter mail are typically used to request these documents. However, the above-described method streamlines the process by automating the process for the payer.

Once the payer has the information they need to adjudicate the claim, the payer clicks on “pay claim” at block 406, which brings up the payment owing (at that time) and reimbursement matrix at block 407. In the illustrated embodiment of FIG. 4, the payer has the option of paying the amount owing in full at block 408, paying only a partial amount of the payment owing at block 409, or disputing the amount at block 410. To facilitate payment, a payment service may be incorporated into the system. The payment system may collect payment from the payer through e-chequing, e-wallet, credit/debit card payment, and the like.

The payer may receive automated notifications and reminders from the system through notifications service 118, which connects to email service 116. Notifications may be sent regarding an upcoming increase in the payment owing (or decrease in the discount), to encourage the payer to avoid further delay of payment.

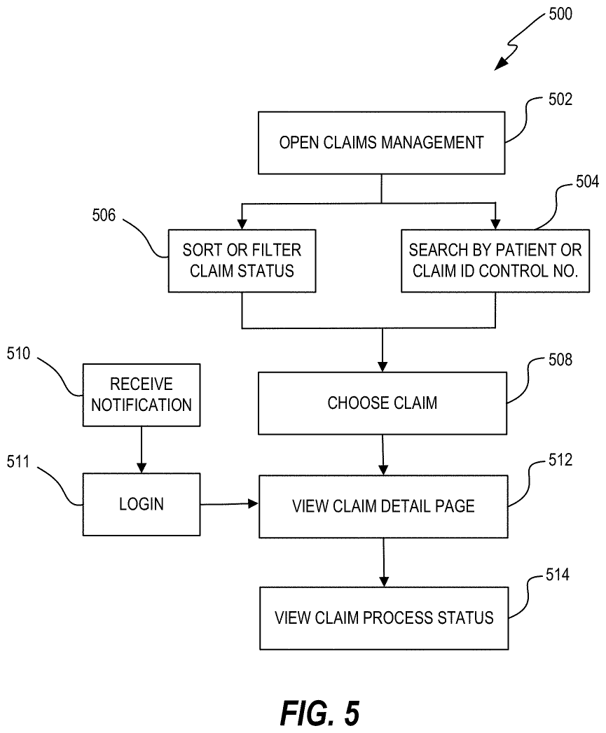

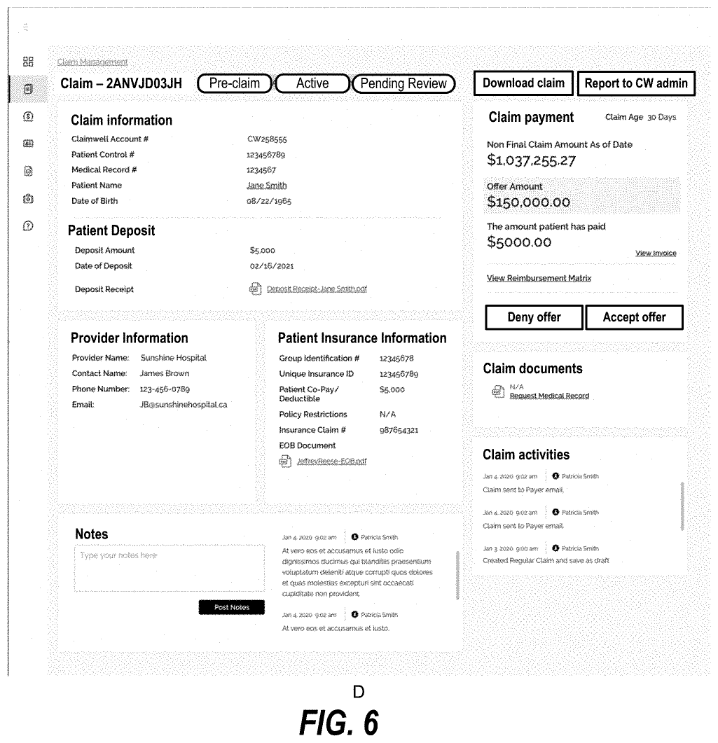

FIG. 5 is a flowchart of a process 500 for tracking a claim by a provider according to one embodiment. After the user has logged into the system, the process 500 begins at block 502 with the user opening the claims management webpage. The user may search by patient or by control number at block 504, and may sort or filter by claim status at block 506. The user selects a claim from the list displayed (block 508), which pulls up a claim detail page (block 512), a representative example of which is shown in FIG. 6. Process 500 finishes at block 514 with the user viewing the claim process status. The claim process status may be included as part of the claim detail page. For example, FIG. 6 shows the claim process status, which includes the claim age (30 days), non-final claim amount as of that date ($1,037,255.27), offer amount ($150,000) (which is an offer to the payer, made by the provider to settle the claim at a substantial discount in cases where the provider believes there is a risk of non-payment by the payer; an offer may be made, for example, based on interim billed charges when a pre-claim is generated), and an amount paid by the patient ($5,000). Not all cases will include an offer as shown, as the provider will decide whether or not it is appropriate to make an offer to settle the claim at a substantial discount in the circumstances.

Some of the advantages provided by the system described herein include price transparency, and the encouragement of fair and reasonable rates through the reimbursement matrix. The reimbursement matrix is determined to be fair and qualified because it is customized by the provider based on their in-network and contracted rates. The use of the reimbursement matrix avoids surprise or unfair medical billing practices, particularly for out-of-network patients who are conventionally often subject to much higher bills for the same service than in-network patients. Even for out-of-network patients, the reimbursement matrix provides the option for the payer to essentially pay the claim at in-network rates, by paying the claim early at the highest discount.

The examples and corresponding diagrams used herein are for illustrative purposes only. Different configurations and terminology can be used without departing from the principles expressed herein. Although the invention has been described with reference to certain specific embodiments, various modifications thereof will be apparent to those skilled in the art without departing from the scope of the invention. For example:
Systems described herein may also be used for electronic health records (EHR), electronic medical records (EMR) and revenue cycle management (RCM).
Blockchain ledger technology may be incorporated for the generation and maintenance of documents and records used in the system.
Machine learning may be used to facilitate identifying which claims should have pre-claims with offers to facilitate better denial management, and identifying pricing patterns that can be made viewable, including cost outcomes based on best, medium, and/or worst case scenarios. Payer rates can be changed with machine learning based on payer performance, without requiring human intervention.
While embodiments described herein are in the context of health care claims management, in other claim management systems the “provider” need not be a health care service provider. The system could be adapted for use with any provider of products or services to a client/customer, who have a need for managing claims for payment by a payor (e.g. the customer, or an insurer of the customer).
In certain alternate embodiments, once a claim has been generated by the provider, a “purchase claim” service displays an offer to buy a claim from the provider before they send the claim to the payer. Typically, this offer discounts the claim amount, but the provider may find the offer to be attractive where the provider has a low risk tolerance for non-payment (e.g. in times of cash flow shortages). If the provider accepts the offer to buy the claim, the operator of the purchase claim service transmits payment to the provider. The operator will then collect payment from the payer.
In certain embodiments, the platform described herein may include a website providing shoppable services. The system gathers information from hospital clients and publishes that on the website (e.g. hospitals offer hip replacement Thursdays or MRI Fridays). Alternately, a patient can log into the system and search for a service, which will bring up options and pricing, and connect the patient with a health care provider.
Certain embodiments may include options for telemedicine, physician concierge (e.g. send a doctor to the patient's hotel room).
Certain embodiments may take into consideration the deductible amount as compared to the cost of the procedure; if the patient requires a smaller procedure than the deductible threshold, the system can provide an alternate option to the patient for payment, rather than submitting the claim to the insurer.
Certain embodiments may have hotel booking options for medical tourism.
Certain embodiments collect and analysis payment information through an analytics service 114. Based on such analysis, providers and/or patients can be informed of payers who have a poor payment record. For example, the system may identify payers that routinely deny or underpay claims and otherwise act in bad faith. The system may recommend more reliable alternatives for claims coverage directly to the patients.

The scope of the claims should not be limited by the illustrative embodiments set forth in the examples, but should be given the broadest interpretation consistent with the description as a whole.

1. A computer-implemented method for managing a claim on behalf of a provider, the method comprising:
(a) receiving the claim via a claim input form provided through a device operated by the provider, the claim comprising patient-identifying information for a patient and claim information;
(b) identifying from the claim a payer associated with the patient;
(c) based on the claim and a payment setting of the payer, generating a reimbursement matrix, and creating a request for payment with the reimbursement matrix; and
(d) transmitting the request for payment to the provider.

2. The method of claim 1 comprising generating the reimbursement matrix by calculating a full claim amount from the claim information, and determining, for each one of a plurality of predetermined dates over a payment period, an outstanding payment amount, wherein the outstanding payment amount is discounted from the full claim amount by an amount that decreases over time.

3. The method of claim 2 comprising sending to the payer, if the claim has not yet been settled, a notification of the outstanding payment amount, on each one of the plurality of predetermined dates.

4. The method of claim 1 comprising collecting payment information from the payer when the payer elects to settle the claim.

5. The method of claim 1 comprising retrieving medical records and/or a medical report for the patient from a health records repository or from the provider.

6. The method of claim 1 comprising extracting a diagnosis code from the claim information, and based on such diagnosis code, looking up associated treatment information in a medical diagnosis repository.

7. The method of claim 2 comprising transmitting the claim to a legal recovery system if the payment period expires without settlement of the claim by the payer.

8. The method of claim 1 comprising retrieving primary care physician records for the patient from a database or the provider.

9. The method of claim 1 wherein the payment setting determines, for each of the predetermined dates over the payment period, an extent to which the claim amount is discounted.

10. A system for managing a claim on behalf of a provider, the system comprising a server configured to:
(a) provide a claim input form for receiving the claim through a device accessed by the provider, the claim comprising patient-identifying information for a patient and claim information;
(b) identify from the claim a payer associated with the patient;
(c) based on the claim and a payment setting of the payer, generate a reimbursement matrix, and create a request for payment with the reimbursement matrix; and
(d) transmit the request for payment to the payer.

11. The system of claim 10 wherein the server comprises a claim calculation module configured to generate the reimbursement matrix by calculating a full claim amount from the claim information, and determine, for each one of a plurality of predetermined dates over a payment period, an outstanding payment amount, wherein the outstanding payment amount is discounted from the full claim amount by an amount that decreases over time.

12. The system of claim 11 comprising a notification service for sending to the payer, if the claim has not yet been settled, a notification of the outstanding payment amount, on each one of the plurality of predetermined dates.

13. The system of claim 10 comprising a payment service for collecting payment information from the payer when the payer elects to settle the claim.

14. The system of claim 10 comprising a medical information retrieval service for retrieving medical records and/or a medical report for the patient from a health records repository or the provider.

15. The system of claim 10 comprising a description generation module for extracting a diagnosis code from the claim information, and based on such diagnosis code, looking up associated treatment information in a medical diagnosis repository.

16. The system of claim 11 comprising a collections service configured to transmit the claim to a legal recovery system if the payment period expires without settlement of the claim by the payer.

17. The system of claim 10 comprising a physician records service for retrieving primary care physician records for the patient from a database or the provider.

18. The system of claim 10 wherein the payment setting determines, for each of the predetermined dates over the payment period, an extent to which the claim amount is discounted.

19. The system of claim 10 wherein the server is configured to obtain and upload benefit documents such as verification of benefits or explanation of benefits.

20. The system of claim 10 wherein the server is configured to receive a request for help and in response to the request, coordinate support through a help desk support web service.

21. The method of claim 1 comprising receiving a pre-claim and sending the pre-claim to the payer while the patient is admitted to hospital or receiving medical care, to assist the payer in reserve setting and payment approval.

22. The method of claim 21 comprising receiving updated interim billed charges from the provider and automatically sending an update of the interim billed charges to the payer.

23. The method of claim 22 comprising providing an option for the payer to deny coverage, and supply documentation to the provider.

24. The method of claim 1 comprising tracking payment performance and payment patterns of payers.

25. The method of claim 1 comprising presenting an offer to purchase the claim from the provider prior to and in lieu of the provider sending the claim to a payer.

26. The method of claim 1 wherein the reimbursement matrix is specific to the claim.
